(19) United States
(12) Patent Application Publication
(10) Pub. No.: US 2011/0118740 A1
(43) Pub. Date: May 19, 2011

(54) Intramedullary Implants Having Variable Fastener Placement

(75) Inventors: Robert A. Rabiner, Tiverton, RI (US); Dennis P. Colleran, North Attleboro, MA (US)

(73) Assignee: IlluminOss Medical, Inc.

(21) Appl. No.: 12/943,544

(22) Filed: Nov. 10, 2010

Related U.S. Application Data
(60) Provisional application No. 61/259,699, filed on Nov. 10, 2009.

(51) Int. Cl. A61B 17/58

(52) U.S. Cl. 606/63

(57) ABSTRACT

Intramedullary implants having variable fastener placement are disclosed herein. In an embodiment, an intramedullary implant includes a non-compliant expandable portion having an outer surface and an inner cavity, wherein a hardened light-sensitive liquid is contained within the inner cavity of the expandable portion; and at least one fastener penetrating the expandable portion at a first location along the outer surface of the expandable portion and into the inner cavity of the expandable portion, wherein the at least one fastener penetrates the expandable portion at a user selected location anywhere along a length of the expandable portion, and wherein the at least one fastener penetrates the expandable portion at any angle relative to the expandable portion.

### RELATED APPLICATIONS

This application claims the benefit of and priority to U.S. Provisional Application Ser. No. 61/259,699, filed Nov. 10, 2009, which is hereby incorporated herein by reference in its entirety for the teachings therein.

### FIELD

The embodiments disclosed herein relate to minimally invasive orthopedic procedures, and more particularly to intramedullary implants having variable fastener placement and methods of using same for fixation of fractured bone segments.

### BACKGROUND

Bone is a living tissue and plays a structural role in the body. A bone fracture is a medical condition in which a bone has cracked or broken. While many fractures are the result of high force impact or stress, bone fracture can also occur as a result of certain medical conditions that weaken the bones, such as osteoporosis, certain types of cancer or osteogenesis imperfecta. The average person sustains two to three fractured bones during the course of a lifetime. Fracture repair is the process of rejoining and realigning the ends of broken bones. Currently there are several approaches to repairing, strengthening and supporting a fractured bone.

### SUMMARY

Intramedullary implants having variable fastener placement and methods of using same are disclosed herein. According to aspects illustrated herein, there is provided an intramedullary implant that includes a non-compliant expandable portion having an outer surface and an inner cavity, wherein a hardened light-sensitive liquid is contained within the inner cavity of the expandable portion; and at least one fastener penetrating the expandable portion at a first location along the outer surface of the expandable portion and into the inner cavity of the expandable portion, wherein the at least one fastener penetrates the expandable portion at a user selected location anywhere along a length of the expandable portion, and wherein the at least one fastener penetrates the expandable portion at any angle relative to the expandable portion. In an embodiment, an intramedullary implant of the present disclosure may be used to align and stabilize fractures of a long bone.

According to aspects illustrated herein, there is provided an intramedullary implant that includes a non-compliant expandable portion having an outer surface and an inner cavity, wherein the non-compliant expandable portion is sized for placement into a medullary canal of a bone; a hardened light-sensitive liquid disposed within the inner cavity of the expandable portion; and at least one fastener penetrating the expandable portion at a first location along the outer surface of the expandable portion and into the inner cavity of the expandable portion, wherein the expandable portion, when placed into a medullary canal of a bone, is configured to accept the at least one fastener at a location anywhere along a length of the expandable portion, and at any angle relative to the expandable portion and to any penetration depth.

According to aspects illustrated herein, there is provided an intramedullary implant kit for use in a medullary canal of a long bone that includes a unit dose of a light-sensitive liquid; a non-compliant expandable portion releasably mounted on an insertion catheter, wherein the insertion catheter has an inner void for passing the light-sensitive liquid to the expandable portion, and an inner lumen; and at least one fastener.

According to aspects illustrated herein, there is provided a method for stabilizing a fractured bone that includes penetrating the fractured bone to gain access to a medullary cavity of the fractured bone; inserting an expandable portion into the medullary cavity of the fractured bone; introducing a light-sensitive liquid monomer into the expandable portion so as to expand the expandable portion, wherein the light-sensitive liquid monomer is introduced into the expandable portion through at least one lumen of an insertion catheter releasably connected to the expandable portion, hardening the light-sensitive liquid monomer within the expandable portion so as to polymerize the light-sensitive liquid monomer; separating the insertion catheter from the expandable portion; and stabilizing the fractured bone, wherein the at least one fastener extends through an outer surface of the fractured bone, through an inner surface of the fractured bone, and into the expandable portion at any location along a length of the expandable portion, at any angle and to any penetration depth relative to the expandable portion.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a side view of an embodiment of a proximal end of an apparatus for insertion of an expandable portion component of an intramedullary implant of the present disclosure to repair a weakened or fractured bone.

FIG. 2 is a side view of an embodiment of a distal end of an apparatus for insertion of an expandable portion component of an intramedullary implant of the present disclosure to repair a weakened or fractured bone.

FIGS. 3A-3B are isometric views of intramedullary implants for repairing a weakened or fractured bone.

FIGS. 4A-4B are isometric views of intramedullary implants for repairing a weakened or fractured bone.

FIGS. 5A-5B are embodiments of an intramedullary implant for repairing a weakened or fractured bone.

FIGS. 6A-6B are embodiments of an intramedullary implant for repairing a weakened or fractured bone.

FIGS. 7A-7B are embodiments of an intramedullary implant of the present disclosure implanted within the intramedullary space of a weakened or fractured bone.

FIG. 8 is a side view of an embodiment of a hole being drilled in a weakened or fractured bone and through an expandable portion for insertion of fasteners through the holes, resulting in an intramedullary implant of the present disclosure.

FIG. 9 is a side view of an embodiment of a fastener being inserted through the weakened or fractured bone and the expandable portion of FIG. 8.

FIGS. 10A-10E show an embodiment of method steps for implanting an expandable portion of an intramedullary device of the present disclosure within the intramedullary space of a weakened or fractured bone.

FIG. 11 illustrates a method for bone fracture stabilization using an intramedullary implant of the present disclosure.

FIG. 12 illustrates a method for bone fracture stabilization using an intramedullary implant of the present disclosure.

FIG. 13 is a schematic illustration of an embodiment of an intramedullary implant kit of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The embodiments disclosed herein relate to minimally invasive orthopedic procedures, and more particularly to intramedullary implants having variable fastener placement and methods of using same for fixation of fractured bone segments. In an embodiment, an intramedullary implant includes a thin-walled, non-compliant, expandable portion having an inner lumen filled with a light-sensitive liquid which has been hardened in situ and at least one fastener having a proximal end and a distal end, wherein the distal end of the fastener penetrates an outer surface of the expandable portion at a user selected insertion spot. In an embodiment, after the distal end of the fastener penetrates the outer surface of the expandable portion, the distal end of the fastener resides within the inner lumen of the expandable portion.

In an embodiment, an intramedullary implant includes a non-compliant expandable portion having an outer surface and an inner cavity, wherein the non-compliant expandable portion is sized for placement into a medullary canal of a bone, a hardened light-sensitive liquid disposed within the inner cavity of the expandable portion, and at least one fastener penetrating the expandable portion at a first location along the outer surface of the expandable portion and into the inner cavity of the expandable portion, wherein the expandable portion, when placed into a medullary canal of a bone, is configured to accept the at least one fastener at a location anywhere along a length of the expandable portion, and at any angle relative to the expandable portion.

In an embodiment, after the distal end of the fastener penetrates the outer surface of the expandable portion, the distal end of the fastener penetrates the outer surface of the expandable portion at a different location than the insertion spot. The fasteners can be inserted at any point along the expandable portion and without regard to orientation, which may allow a surgeon to avoid not only important ligaments/muscle but also avoid critical nerve branches. In an embodiment, no guidance is required to insert the fastener into the expandable portion.

In an embodiment, an intramedullary implant includes a non-compliant expandable portion having an outer surface and an inner cavity, wherein a hardened light-sensitive liquid is contained within the inner cavity of the expandable portion; and at least one fastener penetrating the expandable portion at a first location along the outer surface of the expandable portion and into the inner cavity of the expandable portion, wherein the at least one fastener penetrates the expandable portion at a user selected location anywhere along a length of the expandable portion, and wherein the at least one fastener penetrates the expandable portion at any angle relative to the expandable portion. In an embodiment, an intramedullary implant of the present disclosure is sufficiently designed to induce compression of bone segments during bone fixation. In an embodiment, when the at least one fastener penetrates the hardened expandable portion, compression at the fracture site is induced by angling the fastener to pull the hardened expandable portion and the bone together.

In an embodiment, locking (via a fastener) an intramedullary implant proximally and distally provides rotational and axial stability to the intramedullary implant. When setting a broken bone, the fractured fragments should be aligned with each other so that the fractured edges will mate properly for healing. Intramedullary implants stabilize the fractured fragments and hold them in place for healing. If the intramedullary implant is loose or is able to wobble inside the medullary cavity of the fractured bone, however, the fractured fragments can rotate or shift axially, causing, for example, a rotational displacement about the fracture line, a gap or other discontinuity. In an embodiment, an intramedullary implant of the present disclosure provides rotational stability and resistance to axial migration. As will be described in detail below, the diameter of an intramedullary implant of the present disclosure can be customized during the implantation of the device to achieve a tight fit between the implant and the medullary cavity of the fractured bone. In embodiment, an intramedullary implant of the present disclosure is configured to conform to the internal diameter of the medullary cavity of the fractured bone as well as the curvature of the cavity. In an embodiment, the frictional force on the implant will prevent the bone from rotating on the implant. In an embodiment, the implant may be secured to the bone using fasteners at user selected locations. User selected locations for fastener holes may allow for dynamic compression and shortening while still maintaining rotational stability of the fractured fragments. Because the fasteners may be placed at a short distance from each other, the torsion or torque exerted by the bone on the implant can also be minimized. The fasteners can be placed closer to the proximal/distal sides of the fractured bone, and by doing so, the torque/rotational forces that can be imparted are reduced. The placement of the fasteners closer to the fracture site is patient specific If multiple fasteners are used, the position, depth of penetration and orientation of each fastener relative to a neighboring fastener is independent.

In an embodiment, an intramedullary implant of the present disclosure may be used to align and stabilize fractures of a long bone. In an embodiment, an intramedullary implant of the present disclosure may be used to align and stabilize a long bone including bones selected from the group consisting of metacarpal, femur, tibia, fibula, humerus, ulna, radius, metatarsals, phalanx, phalanges, ribs, spine, vertebrae, clavicle and other bones and still be within the scope and spirit of the disclosed embodiments.

Conventional fixation devices include wires, plates, rods, pins, nails, and fasteners to support the fractured bone directly, as well as the addition of bone cement mixtures, or bone void fillers to the fractured bone. One common device, the intramedullary rod or nail, is implanted into the bone marrow canal in the center of the long bones of the extremities, such as the femur or the tibia. These intramedullary rods are able to share the load with the bone, rather than support the bone entirely, thus allowing patients to use the extremity more quickly. In these conventional fixation devices, the effect is biologic healing, wherein a device has the strength of the bone, not more than the bone, therefore sharing the load across the fracture to stimulate healing.

The use of conventional intramedullary rods results in several disadvantages to both the patient and the staff. For example, intramedullary rods typically contain predrilled holes which are located throughout the rod. To secure an intramedullary rod in place, fasteners, nails or pins are inserted into these holes. Numerous methods and apparatus have been developed to place locking fasteners across both a fractured bone and an implanted intramedullary nail. Nail locking is currently made using either mechanical aiming arms or X-ray guidance. These X-ray guided procedures require the X-ray source positioned such that the X-ray beam is parallel to the axis of the nail hole, increasing X-ray exposure to the patient and the staff. Another disadvantage of the predrilled holes is that the fasteners, nails and pins must be precisely inserted into the holes in order for the rod to be secured. This requires having an aiming system in place to “find” the hole. Moreover, predrilled holes may not be situated in the best locations for securing the rod. As such, fasteners, nails and pins may need to be inserted in sub-optimal places.

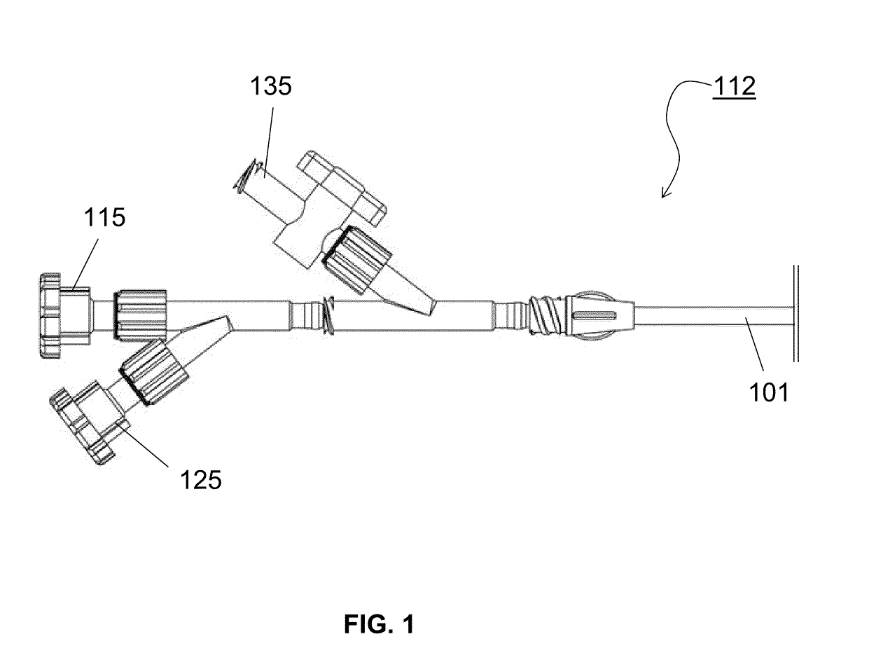

In an embodiment, a flexible insertion catheter may be used for insertion of an expandable portion component of an intramedullary implant of the present disclosure. Generally, such insertion catheters may include an elongated shaft with a proximal end and a distal end, and a longitudinal axis therebetween. FIG. 1 is a side view of an embodiment of a proximal end 112 of a flexible insertion catheter 101 of an apparatus of the present disclosure for insertion of an expandable portion of an intramedullary implant of the present disclosure. In an embodiment, the flexible insertion catheter 101 has an outer diameter from about 2 mm to about 8 mm. In an embodiment, the flexible insertion catheter 101 has an outer diameter from about 3 mm to about 6 mm.

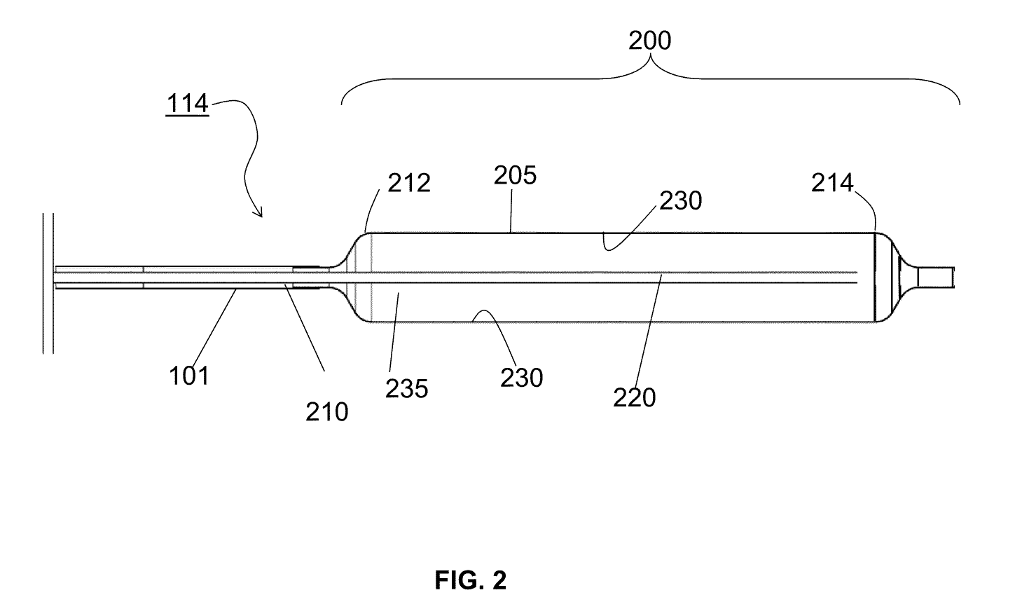

FIG. 2 is a side view of an embodiment of a distal end 114 of the flexible insertion catheter 101. The distal end 114 includes an expandable portion 200 releasably mounted on the flexible insertion catheter 101. The expandable portion 200 has an outer surface 205, an inner surface 230, and an inner cavity 235 defined by the inner surface 230. In an embodiment, the expandable portion 200 is manufactured from a thin-walled, non-compliant (non-stretch/non-expansion) conformable material. The expandable portion 200 may be formed of a pliable, resilient, conformable, and strong material, including but not limited to urethane, polyethylene terephthalate (PET), nylon elastomer and other similar polymers. In an embodiment, the expandable portion 200 of the present disclosure is constructed out of a PET nylon aramet or other non-consumable materials. The expandable portion 200 may be impregnated with a radiopaque material to enhance the visibility of the expandable portion 200. The expandable portion 200 is biocompatible, thus preventing or reducing possible adverse reactions after insertion into a fractured bone. In an embodiment, the expandable portion 200 is made from a material that is non-toxic, non-antigenic and non-immunogenic. The expandable portion 200 includes a proximal area 212 and a distal area 214. The proximal area 212 of the expandable portion 200 is releasable connected to the distal end 114 of the insertion catheter 101.

In an embodiment, a separation area is located at the junction between the expandable portion and the insertion catheter. The separation area may have a stress concentrator. The stress concentrator may be a notch, groove, channel or similar structure that concentrates stress in the separation area. The stress concentrator of the separation area may be notched, scored, indented, pre-weakened or pre-stressed to direct separation of the expandable portion from the elongated shaft of the insertion catheter under specific torsional load. The separation area ensures that there are no leaks of the light-sensitive liquid from the insertion catheter and/or the expandable portion. The separation area seals the expandable portion and removes the insertion catheter by making a break at a known or predetermined site (e.g., a separation area). The separation area may be various lengths and up to about an inch long. In an embodiment, when torque (twisting) is applied to the insertion catheter the shaft of the insertion catheter separates from the expandable portion. The twisting creates a sufficient shear to break the residual hardened light-sensitive and create a clean separation of the expandable portion/insertion catheter interface. In an embodiment, the expandable portion is cut from the insertion catheter using a cutting device.

In an embodiment, the insertion catheter may include multiple inner lumen or voids. For example, as shown in FIG. 2, the insertion catheter includes an inner void 210 for passing a light-sensitive liquid into the expandable portion and an inner lumen 220 for passing a light-conducting fiber (which is not illustrated in FIG. 2). The proximal end 112 of the flexible insertion catheter 101 includes at least two ports. In the embodiment shown in FIG. 1, the proximal end 112 includes three ports 115, 125, and 135. Port 115 can accept, for example, a light-conducting fiber. In an embodiment, the light-conducting fiber is an optical fiber. In an embodiment, the optical fiber has an outer diameter from about 1 mm to about 3 mm. The optical fiber is sized to pass through an inner lumen of the insertion catheter 101. The optical fiber can be made from any material, such as glass, silicon, silica glass, quartz, sapphire, plastic, combinations of materials, or any other material, and may have any diameter. In an embodiment, the optical fiber is made from a polymethyl methacrylate core with a transparent polymer cladding. It should be appreciated that the above-described characteristics and properties of the optical fibers are exemplary and not all embodiments of the present disclosure are intended to be limited in these respects. Port 125 can accept, for example, a syringe housing air or fluid. Port 135 can accept, for example, a syringe housing a light-sensitive liquid. In an embodiment, the light-sensitive liquid is a liquid monomer. In an embodiment, the syringe maintains a low pressure during the infusion and aspiration of the light-sensitive liquid. In an embodiment, the syringe maintains a low pressure of about 10 atmospheres or less during the infusion and aspiration of the light-sensitive liquid.

Light-sensitive liquid can be introduced into the proximal end 112 of the insertion catheter 101 and passes through the inner void 210 of the insertion catheter 101 up into the inner cavity 235 of the expandable portion 200 to move the expandable portion from a deflated state to an inflated state when the light-sensitive liquid is delivered to the expandable portion, in order to form a rigid orthopedic stabilizer. In an embodiment, the light-sensitive liquid is provided as a unit dose. As used herein, the term “unit dose” is intended to mean an effective amount of light sensitive liquid adequate for a single session. By way of example, a unit dose of a light sensitive liquid of the present disclosure for expanding an expandable portion of the present disclosure may be defined as enough light-sensitive liquid to expand the expandable portion so that the expanded expandable portion realigns a fractured bone and/or secures the bone back into an anatomical position. The amount of realigning may vary somewhat from user to user. Thus, a user using a unit dose may have excess light-sensitive liquid left over. It is desirable to provide enough light-sensitive liquid that even the above-average user will have an effective amount of realignment. In an embodiment, a unit dose of a light-sensitive liquid of the present disclosure is contained within a container. In an embodiment, a unit dose of a light-sensitive liquid of the present disclosure is contained in an ampoule. In an embodiment, the expandable portion is sufficiently shaped to fit within a space or a gap in a fractured bone. In an embodiment, the light-sensitive liquid can be delivered under low pressure via a standard syringe attached to the port 135. The light-sensitive liquid can be aspirated and reinfused as necessary, allowing for adjustments to the expandable portion. These properties allow a user to achieve maximum fracture reduction prior to activating a light source and converting the liquid monomer into a hard polymer.

A light-conducting fiber communicating light from the light source can be introduced into the proximal end 112 of the insertion catheter 101 through port 115 and passes within an inner lumen of the insertion catheter 101 up into the expandable portion. In an embodiment, the light source emits frequency that corresponds to a band in the vicinity of 390 nm to 770 nm, the visible spectrum. In an embodiment, the light source emits frequency that corresponds to a band in the vicinity of 410 nm to 500 nm. In an embodiment, the light source emits frequency that corresponds to a band in the vicinity of 430 nm to 450 nm. The light-sensitive liquid remains a liquid monomer until activated by the light-conducting fiber (cures on demand). In an embodiment, the liquid monomer is exposed to an appropriate frequency of light and intensity to cure the monomer inside the expandable portion and form a rigid structure. In an embodiment, the liquid monomer is exposed to electromagnetic spectrum that is visible (frequency that corresponds to a band in the vicinity of 390 nm to 770 nm). In an embodiment, the liquid monomer is radiolucent, which permit x-rays to pass through the liquid monomer. Radiant energy from the light source is absorbed and converted to chemical energy to quickly (e.g., cured in about five seconds to about five minutes) polymerize the monomer. This cure affixes the expandable portion in an expanded shape. A cure may refer to any chemical, physical, and/or mechanical transformation that allows a composition to progress from a form (e.g., flowable form) that allows it to be delivered through the inner void in the insertion catheter 101, into a more permanent (e.g., cured) form for final use in vivo. For example, “curable” may refer to uncured composition, having the potential to be cured in vivo (as by catalysis or the application of a suitable energy source), as well as to a composition in the process of curing (e.g., a composition formed at the time of delivery by the concurrent mixing of a plurality of composition components).

Additives may be included in light-sensitive liquids, including, but not limited to, drugs (for example, antibiotics), proteins (for example, growth factors) or other natural or synthetic additives (for example, radiopaque or ultrasonically active materials). In an embodiment, the viscosity of the light-sensitive liquid has a viscosity of about 1000 cP or less. In an embodiment, the light-sensitive liquid has a viscosity ranging from about 650 cP to about 450 cP. The expandable portion may be inflated, trial fit and adjusted as many times as a user wants with the light-sensitive liquid, up until the light source is activated, when the polymerization process is initiated. Because the light-sensitive liquid has a liquid consistency and is viscous, the light-sensitive liquid may be delivered using low pressure delivery and high pressure delivery is not required, but may be used.

In an embodiment, a contrast material may be added to the light-sensitive liquid without significantly increasing the viscosity. Contrast materials include, but are not limited to, barium sulfate, tantalum, or other contrast materials known in the art. The light-sensitive liquid can be introduced into the proximal end of the insertion catheter and passes within the inner void of the insertion catheter up into an inner cavity of the expandable portion to change a thickness of the expandable portion without changing a width or depth of the expandable portion. In an embodiment, the light-sensitive liquid is delivered under low pressure via the syringe attached to the port. The light-sensitive liquid can be aspirated and reinfused as necessary, allowing for thickness adjustments to the expandable body prior to activating the light source and converting the liquid monomer into a hard polymer. Low viscosity allows filling of the intramedullary implant through a very small delivery system.

One or more radiopaque markers or bands may be placed at various locations along the expandable portion 200 and/or the insertion catheter 101. A radiopaque ink bead may be placed at a distal end of the expandable portion for alignment of the apparatus during fluoroscopy. The one or more radiopaque bands and radiopaque ink bead, using radiopaque materials such as barium sulfate, tantalum, or other materials known to increase radiopacity, allows a medical professional to view the apparatus using fluoroscopy techniques. The one or more radiopaque bands also provide visibility during inflation of the expandable portion to determine the precise positioning of the expandable portion during placement and inflation. The one or more radiopaque bands permit visualization of any voids that may be created by air that gets entrapped in the expandable portion. The one or more radiopaque bands permit visualization to preclude the expandable portion from misengaging or not meeting a bone due to improper inflation to maintain a uniform expandable portion/bone interface.

In an embodiment, the expandable portion 200 can have a length greater than about 300 mm and a diameter greater than about 14 mm. In such embodiments, there is the potential that during the curing of the light-sensitive liquid, a far distal area 214 of the expandable portion 200 will exhibit a shrinkage upon cure of about 2 to about 3 percent, while a proximal area 212 of the expandable portion 200 is being cured. In an embodiment, to prevent this from transpiring, the inner lumen 220 of the expandable portion 200 can be pressurized by virtue of the infusion of either air or other fluids (saline, water) through port 125 at the proximal end 112 of the insertion catheter 101. The infusion will cause internal diameter pressure against the light-sensitive liquid contained within the inner cavity 235 of the expandable portion 200 so that during the curing process, the pressure keeps the light-sensitive liquid pressurized, and up in contact with inner surface 230 of the expandable portion 200. When the light-conducting fiber is inserted within the inner lumen 220 and the light-sensitive liquid is infused—the extra space is pressed down on the inner lumen 220. In an embodiment, an expandable portion of the present disclosure has a diameter ranging from about 4 mm to about 30 mm. In an embodiment, an expandable portion of the present disclosure has a length ranging from about 20 mm to about 300 mm. An expandable portion of the present disclosure may be round, flat, cylindrical, oval, rectangular or any desired shape for a given application. In an embodiment, an expandable portion of the present disclosure has a diameter of about 4 mm and a length of about 30 mm. In an embodiment, an expandable portion of the present disclosure has a diameter of about 5 mm and a length of about 40 mm. In an embodiment, an expandable portion of the present disclosure has a diameter of about 6 mm and a length of about 30 mm. In an embodiment, an expandable portion of the present disclosure has a diameter of about 6 mm and a length of about 40 mm. In an embodiment, an expandable portion of the present disclosure has a diameter of about 6 mm and a length of about 50 mm. In an embodiment, an expandable portion of the present disclosure has a diameter of about 7 mm and a length of about 30 mm. In an embodiment, an expandable portion of the present disclosure has a diameter of about 7 mm and a length of about 40 mm. In an embodiment, an expandable portion of the present disclosure has a diameter of about 7 mm and a length of about 50 mm.

In an embodiment, an outer surface of an expandable portion of the present disclosure is resilient. In an embodiment, an outer surface of an expandable portion of the present disclosure is substantially even and smooth. In an embodiment, an outer surface of an expandable portion of the present disclosure is not entirely smooth and may have some small bumps or convexity/concavity along the length. In an embodiment, an outer surface of an expandable portion of the present disclosure may have ribs, ridges, projections, bumps or other shapes. In an embodiment, the ribs, ridges, projections, bumps, or other shapes on the rough or uneven outer surface of the expandable portion improve penetration of the at least one fastener into the expandable portion. In an embodiment, the ribs, ridges, projections, bumps, or other shapes on the rough or uneven outer surface of the expandable portion improve penetration of the at least one fastener into the expandable portion anywhere along a length of the expandable portion. In an embodiment, the ribs, ridges, projections, bumps, or other shapes on the rough or uneven outer surface of the expandable portion increase friction between the outer surface of the expandable portion and the at least one fastener so as to reduce slippage of the at least one fastener as the at least one fastener is driven towards the outer surface of the expandable portion. In an embodiment, the ribs, ridges, projections, bumps, or other shapes on the rough or uneven outer surface of the expandable portion interacts with a threaded portion of the at least one fastener so as to improve penetration and fastening of the at least one fastener into the expandable portion. In an embodiment, the ribs, ridges, projections, bumps, or other shapes on the rough or uneven outer surface of the expandable portion interact with a tip of the at least one fastener to improve the wedge ability of the tip of the fastener so as to decrease the driving force needed to penetrate the expandable portion. In an embodiment, an outer surface of an expandable portion of the present disclosure has an uneven geometry. In an embodiment, an outer surface of an expandable portion of the present disclosure has a textured surface which provides one or more ridges that allow grabbing. In an embodiment, the one or more ridges on the textured surface of the expandable portion allow grabbing of the at least one fastener so as to improve the penetration of the at least one fastener into the expandable portion. In an embodiment, the one or more ridges on the textured surface of the expandable portion allow grabbing of bone so as to improve adhesion between the expandable portion and bone as regenerating bone grows onto the outer surface of the expandable portion. In an embodiment, abrasively treating an outer surface of an expandable portion of the present disclosure for example via chemical etching or air propelled abrasive media improves the connection and adhesion between the outer surface of the expandable portion and a bone. The surfacing may significantly increase the amount of surface area that comes in contact with the bone resulting in a stronger grip. In an embodiment, the textured surface promotes bone growth onto the expandable portion. In an embodiment, the textured surface promotes bone growth of regenerating bone onto the outer surface of the expandable portion by grabbing the regenerating bone as it grows. In an embodiment, an expandable portion of the present disclosure is made by extruding material into a tube shape, and then forming the tube into a balloon. When forming the tube into the balloon, the balloon can be, for example, pre-stamped or milled to include a desired design, desired shape or surface modification. Then, the tube is heated and radially expanded via compressed air for a specific amount of time. The formed balloon is cooled and includes the desired design, desired shape or surface modification.

In an embodiment, an expandable portion of the present disclosure has an outer surface that is coated with materials such as drugs, bone glue, proteins, growth factors, or other coatings. For example, after a minimally invasive surgical procedure an infection may develop in a patient, requiring the patient to undergo antibiotic treatment. An antibiotic drug may be added to an outer surface of an expandable portion of the present disclosure to prevent or combat a possible infection. Proteins, such as, for example, bone morphogenic protein or other growth factors have been shown to induce the formation of cartilage and bone. In an embodiment, a growth factor is added to an outer surface of an expandable portion of the present disclosure to help induce the formation of new bone. In an embodiment, as the formation of new bone is induced the new bone interacts with a textured outer surface of the expandable portion so that new bone is formed onto the textured outer surface of the expandable portion. Due to the lack of thermal egress of light-sensitive liquid in an expandable portion of the present disclosure, the effectiveness and stability of the coating is maintained.

In an embodiment, a stiffness of any of the expandable portion of the present disclosure can be increased due to the presence of external stiffening members or internal stiffening members. In an embodiment, a wrapping, sheathing or an attachment of Nitonol or other metallic memory-type metal piece(s) are aligned in a longitudinal fashion, with multiple rods being placed circumferentially around the expandable portion so as to have these metallic pieces change their configuration under a temperature change. In an embodiment, an inner surface of the metallic pieces (those surfaces that are in contact with the external circumferential surface of the intramedullary implant) are polished to increase internal reflection of the light from the light-conducting fiber. The metallic pieces are designed to be load-bearing shapes. In an embodiment, the metallic pieces have a low profile and can handle large loads. In an embodiment, metallic pieces may be positioned on the external circumferential surface of an expandable portion. The metallic pieces can be aligned in a longitudinal fashion, circumferentially around the expandable portion and can be interconnected with one another via connecting means such as wires. The wires will help hold the longitudinal metallic pieces in position. In an embodiment, the metallic pieces expand to increase the strength of the hardened expandable portion. In an embodiment, the metallic pieces contract to increase the strength of the hardened expandable portion. In an embodiment, metallic pieces are positioned on an internal circumferential surface of an expandable portion. In an embodiment, two metallic memory-type metal wires, such as nitonol, are positioned within an expandable portion. Heat from a light-conducting fiber makes the metal wires get smaller, tensioning the hardened expandable portion. In an embodiment, heat from a light-conducting fiber and reaction with the polymerization process, makes the metal wires get smaller, tensioning the hardened expandable portion. In an embodiment, an expandable portion is wrapped with a plurality of flat metallic plates that move into a corrugated or other shape upon a temperature change to increase the strength of the previously flat metal plate into a shape capable of handling a load. In an embodiment, the metals are rectangular, semicircular, hexagonal, or triangular in section, although not all embodiments are limited to these shapes.

An expandable portion typically does not have any valves. One benefit of having no valves is that the expandable portion may be inflated or deflated as much as necessary to assist in the fracture reduction and placement. Another benefit of the expandable portion having no valves is the efficacy and safety of the implant. Since there is no communication passage of light-sensitive liquid to the body there cannot be any leakage of liquid because all the liquid is contained within the expandable portion. In an embodiment, a permanent seal is created between the expandable portion that is both hardened and affixed prior to the insertion catheter 101 being removed. The expandable portion may have valves, as all of the embodiments are not intended to be limited in this manner.

In an embodiment, an expandable portion of the present disclosure includes a pathway sufficiently designed for passing a cooling medium. Once the expandable portion is expanded, a cooling media may be delivered within (via an internal lumen) or around (via external tubing) the expandable portion in order to prevent the possibility of overheating. Medium used for cooling includes, but is not limited to, gases, liquids and combinations thereof. Examples of gases include, but are not limited to, inert gases and air. Examples of liquids include, but are not limited to, water, saline, saline-ice mixtures, liquid cryogen. In an embodiment, the cooling media is water. The cooling media can be delivered to the expandable portion at room temperature or at a cooled temperature. In an embodiment, the cooling media improves the numerical aperture between that of the light-conducting fiber and the inner lumen for the light-conducting fiber because any air existing between the light-conducting fiber and the material of the expandable portion is taken away so as to improve light transmission. Therefore, the light transmission will be light-conducting fiber-cooling media-expandable portion-light-sensitive liquid as opposed to light-conducting fiber-air-expandable portion-light-sensitive liquid. In an embodiment, the cooling media transmitted through the inner lumen of the expandable portion takes away extraneous heat. In an embodiment, no cooling media is used.

In an embodiment, the inner lumen of the expandable portion penetrates through a distal end of the expandable portion for cooling through the length of the expandable portion. In an embodiment, the inner lumen has a return flow path for cooling. In an embodiment, the inner lumen is pressurized to move the cooling media in the inner lumen. In an embodiment, the expandable portion has external helical tubing for providing cooling media to the expandable portion.

In an embodiment, a light-conducting fiber can be introduced into the inner lumen of the expandable portion and activated to cure the light-sensitive liquid, while a cooling medium may flow through the inner lumen and out the distal end of the expandable portion.

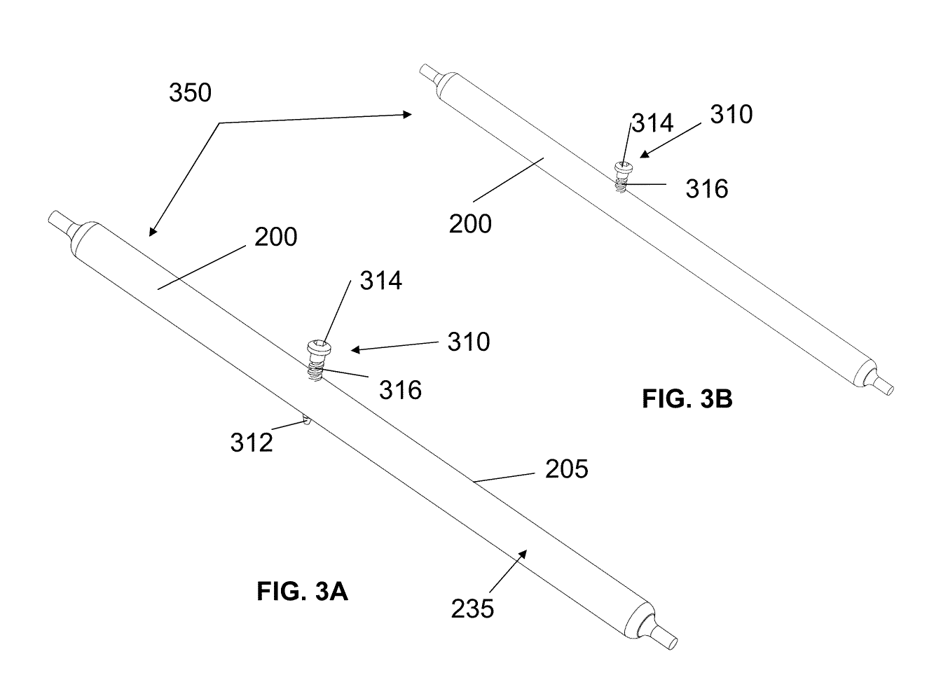

FIGS. 3A-6B show various embodiments of intramedullary implants of the present disclosure. FIGS. 3A and 3B are isometric views of an intramedullary implant 350 that includes expandable portion 200 having the outer surface 205 and the inner cavity 235, which contains a hardened light-sensitive liquid. When the expandable portion 200 is inflated with light-sensitive liquid, it can conform to the internal diameter of a medullary cavity of a fractured bone in which it is placed, and also can conform to the curvature of the medullary cavity so that the curves/compound shapes of the fractured bone are matched by the expandable portion 200. A fastener 310 can be inserted through the expandable portion 200 anywhere along the length of the expandable portion 200, at any angle, and to any desired depth. The fastener 310 does not have to be positioned in the middle of the expandable portion 200 (e.g., a center line drill hole does not need to be made) but in any location where the fastener 310 transits the cortex of the bone so as to act as a wedge or other keyway, precluding rotation of the expandable portion 200 or bone. The fastener 310 can be angled into the expandable portion 200 so as to cause compression between the proximal and distal sections of the expandable portion 200. In an embodiment, the fastener 310 acts to pull the bone fragment pieces together, which can lead to improved alignment. The fastener 310 may include a proximal end 314, a distal end 312, and a body 316 therebetween. In an embodiment, the fastener 310 is a screw. The fastener 310 may also be a pin, peg, nail, bolt, wood fastener, lag fastener, double ended fastener, cap fastener, or any other device, by any name that can generally be used to attach to an object or to connect objects, or any other commercially available type of fastener as the present disclosure is not intended to be limited in this manner. In an embodiment, the fastener has threads to engage bone and the hardened expandable portion. The reference to the “head” of a fastener is intended to refer to the end, or portion of the fastener, that is closer to where force would be applied that imparts motion to the fastener. The “head” may also refer to that portion away from the portion that first enters an object. Some fasteners are commonly referred to as being “headless;” because they do not have a pronounced end portion that distinguishes the end portion from the rest of the fastener. Accordingly, the reference to a “head” of the fastener is not meant to limit the present disclosure in any way to a fastener with one portion that is distinguishable from the rest of the fastener.

In the embodiment shown in FIG. 3A, the fastener 310 may be inserted in a manner such that the distal end 312 of the fastener 310 extends beyond the outer surface 205 of the expandable portion 200. Extending the fastener beyond the outer surface 205 of the expandable portion 200 is known as biocortical purchase. By extending beyond the expandable portion 200, the distal end 312 may help to secure the expandable portion 200 to the fractured bone (not shown) on both sides of the expandable portion and increase torsional strength and axial strength of the intramedullary implant. In the embodiment shown in FIG. 3B, the fastener 310 may be inserted in a manner such that the distal end (not shown) remains within the inner cavity 235 of the expandable portion 200. By allowing the distal end 312 to remain within the lumen, risk of injury to soft tissue, ligamentous structures and nerves may decrease. The intramedullary implant 350 is sufficiently strong, but not so strong as to preclude biologic healing. In an embodiment, the implanted intramedullary implant 350 allows for micro-motion which can promote callus formation. In an embodiment, a bone plate (not shown) may be used with the intramedullary implant 350 to further stabilize the weakened or fractured bone. In an embodiment, the bone plate may receive one or more of fasteners 310 to support the weakened or fractured bone.

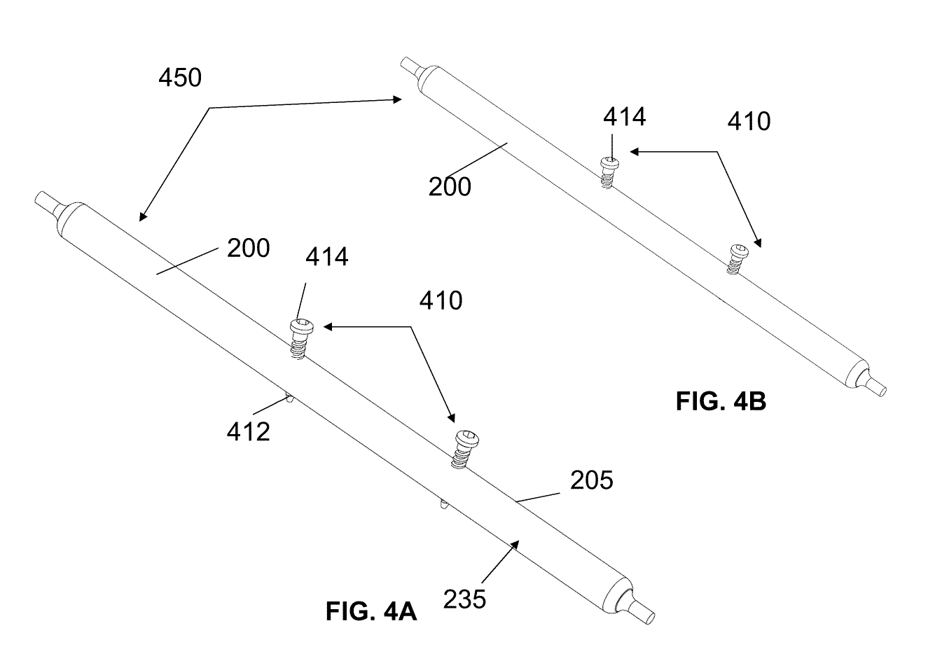

FIG. 4A and FIG. 4B are isometric views of an intramedullary implant 450 that includes expandable portion 200 having outer surface 205 and inner cavity 235, which contains a hardened light-sensitive liquid. When the expandable portion 200 is inflated with light-sensitive liquid, it can conform to the internal diameter of a medullary cavity of a fractured bone in which it is placed, and also can conform to the curvature of the medullary cavity so that the curves/compound shapes of the fractured bone are matched by the expandable portion 200. Two fasteners 410 can be inserted through the expandable portion 200. The fasteners 410 can be positioned anywhere along the length of the expandable portion 200, at any angle, and to any desired depth. The fasteners 410 do not have to be positioned in the middle of the expandable portion 200 (e.g., a center line drill hole does not need to be made) but in any location where the fasteners 410 transit the cortex of the bone so as to act as a wedge or other keyway, precluding rotation of the expandable portion 200 or the bone fragments. The fasteners 410 can be angled into the expandable portion 200 so as to cause compression between the proximal and distal sections of the expandable portion 200. In an embodiment, the fasteners 410 acts to pull the bone fragment pieces together, which can lead to improved alignment. The fasteners 410 may include a proximal end 414, a distal end 412, and a body therebetween. In the embodiment shown in FIG. 4A, the fasteners 410 may be inserted in such a manner that the distal ends 412 of the fasteners 410 extend beyond the outer surface 205 the expandable portion 200. By extending beyond the expandable portion 200, the distal ends 412 may help to secure the expandable portion 200 to the fractured bone (not shown).

In the embodiment shown in FIG. 4B, the fasteners 410 may be inserted in a manner such that the distal ends (not shown) remain within the inner cavity of the expandable portion 200. It will be understood that the number fasteners inserted into the expandable portion 200 as the present disclosure may vary, and thus the disclosure is not intended to be limiting in this manner. In an embodiment, the expandable portion 200 has three fasteners inserted through the expandable portion 200. In an embodiment, the expandable portion 200 has four fasteners inserted through the expandable portion 200. The intramedullary implant 450 is sufficiently strong, but not so strong as to preclude biologic healing. The intramedullary implant 450 is sufficiently strong, but not so strong as to preclude biologic healing. In an embodiment, the implanted intramedullary implant 450 allows for micro-motion which can promote callus formation. In an embodiment, a bone plate (not shown) may be used with the intramedullary implant 450 to further stabilize the weakened or fractured bone. In an embodiment, the bone plate may receive one or more of fasteners 410 to support the weakened or fractured bone.

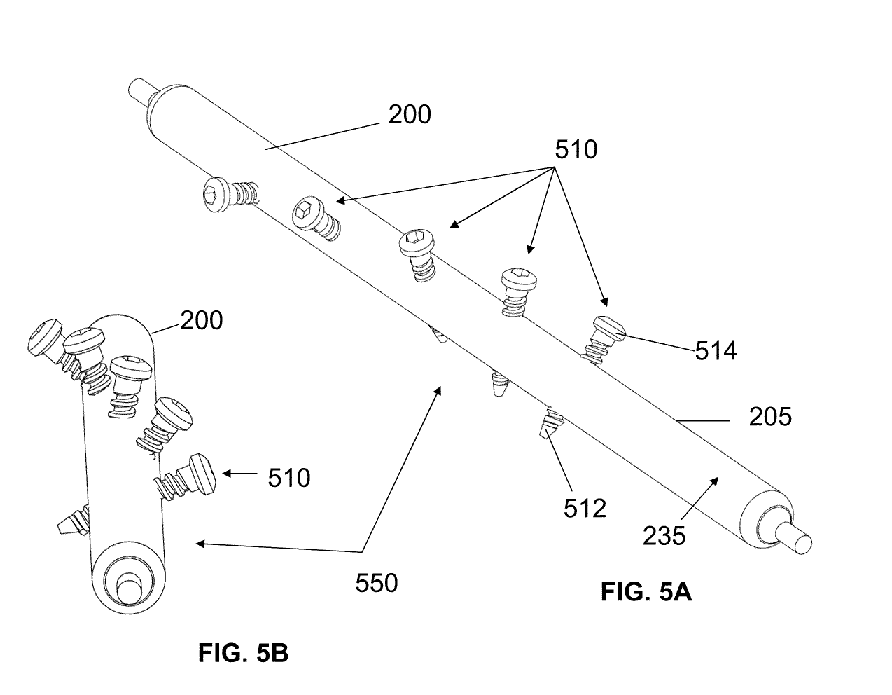

FIG. 5A and FIG. 5B are embodiments of an intramedullary implant 550 that includes expandable portion 200 having outer surface 205 and inner cavity 235, which contains a hardened light-sensitive liquid. When the expandable portion 200 is inflated with light-sensitive liquid, it can conform to the internal diameter of a medullary cavity of a fractured bone in which it is placed, and also can conform to the curvature of the medullary cavity so that the curves/compound shapes of the fractured bone are matched by the expandable portion 200. Five fasteners 510 can be inserted through the expandable portion 200. The fasteners 510 can be positioned anywhere along the length of the expandable portion 200, at any angle, and to any desired depth. The fasteners 510 do not have to be positioned in the middle of the expandable portion 200 (e.g., a center line drill hole does not need to be made) but in any location where the fasteners 510 transit the cortex of the bone so as to act as a wedge or other keyway, precluding rotation of the expandable portion 200 or the bone fragments. The fasteners 510 can be angled into the expandable portion 200 so as to cause compression between the proximal and distal sections of the expandable portion 200. In an embodiment, the fasteners 510 acts to pull the bone fragment pieces together, which can lead to improved alignment. The fasteners 510 may include a proximal end 514, a distal end 512, and a body therebetween. In an embodiment, the five fasteners 510 may be positioned in a spherical orientation such that each fastener 510 is situated about fifteen degrees from an adjacent fastener 510. The orientation of the fasteners 510 may help secure the expandable portion 200 to the fractured bone and reduce the rotational ability of the expandable portion 200. It is important to note, however, that the fasteners 510 may be positioned in any orientation. In an embodiment, the fasteners 510 may be positioned randomly about the expandable portion 200. In an embodiment, the five fasteners 510 may be separated from an adjacent fastener 510 by about 5 mm. Placement of the fasteners 510 closer to one another may increase the stability and reduce the rotational ability of the expandable portion 200 within the fractured bone. Of course, the fasteners 510 can be placed at any distance from one another as the present disclosure is not intended to be limited in this manner. In the embodiment shown in FIG. 5A and FIG. 5B, the fasteners 510 may be inserted in such a manner that the distal ends 512 of the fasteners 510 extend beyond the outer surface 205 of the expandable portion 200. By extending beyond the expandable portion 200, the distal ends 512 may help secure the expandable portion 500 to the fractured bone (not shown) on both sides of the expandable portion 200. The intramedullary implant 550 is sufficiently strong, but not so strong as to preclude biologic healing. In an embodiment, the implanted intramedullary implant 550 allows for micro-motion which can promote callus formation. In an embodiment, a bone plate (not shown) may be used with the intramedullary implant 550 to further stabilize the weakened or fractured bone. In an embodiment, the bone plate may receive one or more of fasteners 510 to support the weakened or fractured bone.

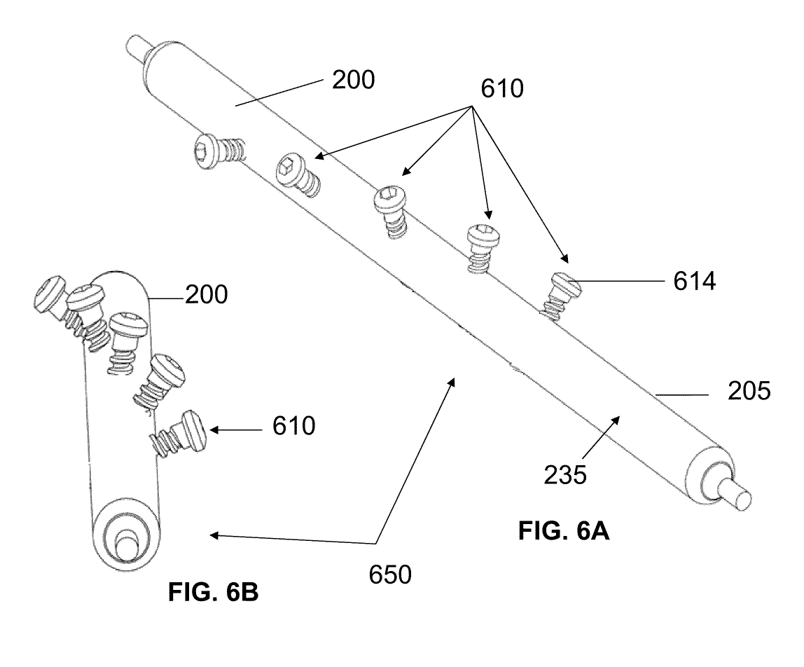

FIG. 6A and FIG. 6B are isometric views of an intramedullary implant 650 that includes expandable portion 200 having outer surface 205 and inner cavity 235, which contains a hardened light-sensitive liquid. When the expandable portion 200 is inflated with light-sensitive liquid, it can conform to the internal diameter of a medullary cavity of a fractured bone in which it is placed, and also can conform to the curvature of the medullary cavity so that the curves/compound shapes of the fractured bone are matched by the expandable portion 200. Five fasteners 610 can be inserted through the expandable portion 200. The fasteners 610 can be positioned anywhere along the length of the expandable portion 200, at any angle, and to any desired depth. The fasteners 610 do not have to be positioned in the middle of the expandable portion 200 (e.g., a center line drill hole does not need to be made) but in any location where the fasteners 610 transit the cortex of the bone so as to act as a wedge or other keyway, precluding rotation of the expandable portion 200 or the bone fragments. The fasteners 610 can be angled into the expandable portion 200 so as to cause compression between the proximal and distal sections of the expandable portion 200. In an embodiment, the fasteners 610 acts to pull the bone fragment pieces together, which can lead to improved alignment. The fasteners 610 may include a proximal end 614, a distal end (not visible), and a body therebetween. In an embodiment, the five fasteners 610 may be positioned in a spherical orientation such that each fastener 610 is situated about fifteen degrees from an adjacent fastener 610. The orientation of the fasteners 610 may help secure the expandable portion 200 to the fractured bone and reduce the rotational ability of the expandable portion 200. It is important to note, however, that the fasteners 610 may be positioned in any orientation. In an embodiment, the fasteners 610 may be positioned randomly about the expandable portion 200. In an embodiment, the five fasteners 610 may be separated from an adjacent fastener 610 by about 5 mm. Placement of the fasteners 610 closer to one another may increase the stability and reduce the rotational ability of the expandable portion 200 within the fractured bone. Of course, the fasteners 610 can be placed at any distance from one another as the present disclosure is not intended to be limited in this manner. In contrast to the embodiments shown in FIG. 5A and FIG. 5B, in the embodiment shown in FIG. 6A and FIG. 6B, the fasteners 610 may be inserted in such a manner that the distal ends of the fasteners remain within the inner cavity 615 of the expandable portion 200. In such embodiments, the implant 650 is secured to the fractured bone only on one side. The intramedullary implant 650 is sufficiently strong, but not so strong as to preclude biologic healing. In an embodiment, the implanted intramedullary implant 650 allows for micro-motion which can promote callus formation. In an embodiment, a bone plate (not shown) may be used with the intramedullary implant 650 to further stabilize the weakened or fractured bone. In an embodiment, the bone plate may receive one or more of fasteners 610 to support the weakened or fractured bone.

Fasteners can be inserted anywhere along the length of an expandable portion of the present disclosure as there are no predrilled holes that determine where the fasteners must be inserted. The fasteners can also be inserted through an expandable portion from any direction and from any angle, independently of each other. This variable placement of fasteners from multiple directions and from multiple angles may help secure an expandable portion in place, reduce rotational ability of the implant, and increase the torsional and axial strength of the implant. In an embodiment, adding 3 mm fasteners to an 8×80 mm intramedullary implant may increase the torsional strength from approximately 8.5 inches per pound to approximately 21.2 inches per pound. It is importance to note that the torsional strength may be a function of bone strength, bone size, bone geometry, fastener size, fastener quality and other characteristics. The fasteners can also be inserted through an expandable portion to any desired depths, independently of each other. For example, although in the embodiments shown in FIGS. 3A-6B, all fasteners either extend beyond the outer surface of the expandable portion or remain within the inner cavity of the expandable portion, there may be embodiments in which only some fasteners will extend beyond an outer surface of the expandable portion, while other fasteners will remain in the inner cavity of the expandable portion.

In an embodiment, a fastener may be inserted at approximately a ninety degree angle to an expandable portion. In an embodiment, a fastener may be inserted at an angle of less than approximately ninety degrees to an expandable portion. In an embodiment, a fastener may be inserted at an angle of more than approximately ninety degrees to an expandable portion. Fasteners can also be inserted from multiple directions and from multiple angles. In an embodiment, fasteners may be inserted from approximately opposite sides allowing them to be approximately parallel to one another. In an embodiment, fasteners may be inserted from approximately ninety degree angles to one another allowing them to be approximately perpendicular to one another. In an embodiment, fasteners may be inserted from less or more than approximately ninety degree angles to one another. The fasteners can also be inserted to any desired depth. In an embodiment, the fasteners can be inserted in such a manner that the distal ends extend beyond an outer surface of the expandable portion. In an embodiment, the distal end of the fasteners extending beyond an outer surface of the expandable portion can be received by a bone plate. In an embodiment, the fasteners can be inserted in such a manner that the distal ends remain within a lumen of an expandable portion. In an embodiment, the fasteners can be inserted in such a manner that a portion of the proximal end of the fastener penetrates the bone plate and the distal end remains within a lumen of the expandable portion. The proximity of the fasteners from one another can also vary depending on the specific application. Increasing the proximity of the fasteners to one another may help secure the expandable portion in place and reduce rotational ability of the intramedullary implant.

By inserting the fastener anywhere along the length of an expandable portion, at any angle and to any desired depth, an intramedullary implant of the present disclosure may increase a user's control over determining optimal fastener placement and reducing or eliminating the need for aiming systems to guide the fastener into place. Accordingly, the user is able to determine the optimal placement of fasteners based on each patient's specific situation rather than on the predrilled holes. For instance, certain situations may require having more fasteners placed in closer proximity while other situations may require fewer fasteners spaced further apart. By increasing user control of fastener placement, an intramedullary implant of the present disclosure may also reduce the likelihood of harming soft tissue, nerves, ligaments or muscles during placement. In conventional intramedullary implants, there may be a risk of injury to tissue, radial or ulna nerves, ligaments or muscles associated with inserting fasteners into predetermined spaces. Predetermined spaces require specific fastener location and orientation and may not accommodate a large variation in patient anatomies. As a result, injuries, including pain and loss of function, to surrounding tissue, nerves, ligaments and/or muscles may occur.

In an embodiment, a method for stabilizing a fractured bone includes penetrating the fractured bone to gain access to a medullary cavity of the fractured bone, inserting an expandable portion into the medullary cavity of the fractured bone, introducing a light-sensitive liquid into the expandable portion through at least one lumen of an insertion catheter connected to the expandable portion, separating the insertion catheter from the expandable portion at a predetermined site, and stabilizing the fractured bone by placing one or more fasteners through the fractured bone and into the expandable portion, wherein the fastener is placed into the expandable portion at any location along the length of the expandable portion, and at any angle and to any penetration depth relative to the expandable portion. In an embodiment, the ability to deliver the at least one fastener anywhere along the length of the expandable portion reduces the time of the procedure, compared to a similar procedure using conventional fixation devices. In an embodiment, the ability to deliver the at least one fastener anywhere along the length of the expandable portion reduces the requirement/need for additional incremental radiation exposure to the patient and the doctor.

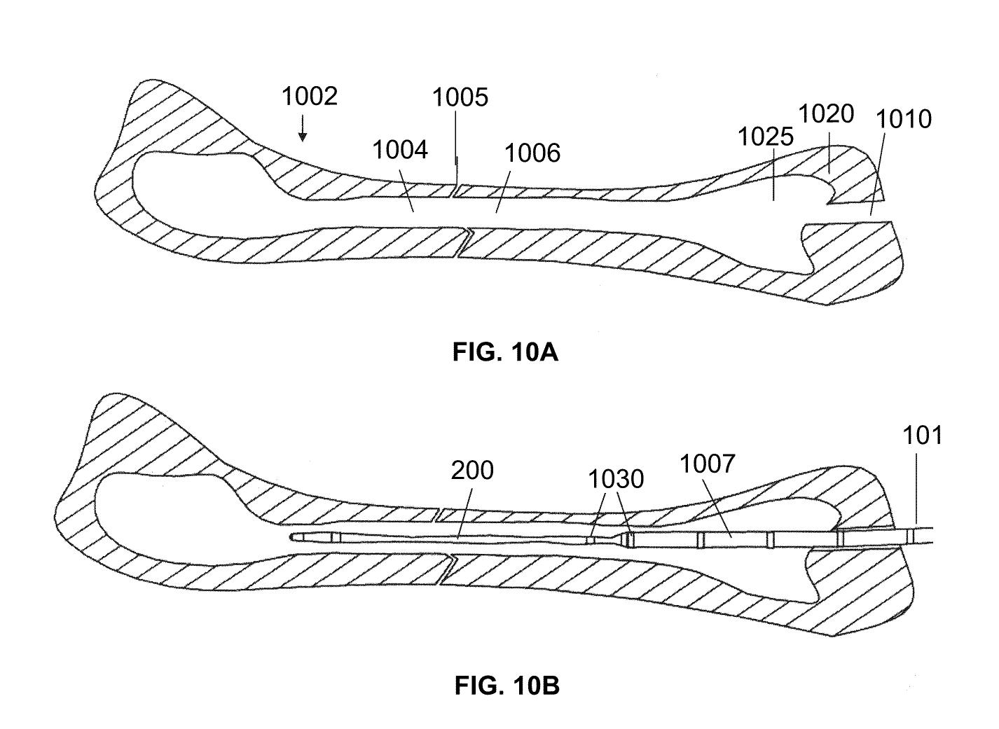

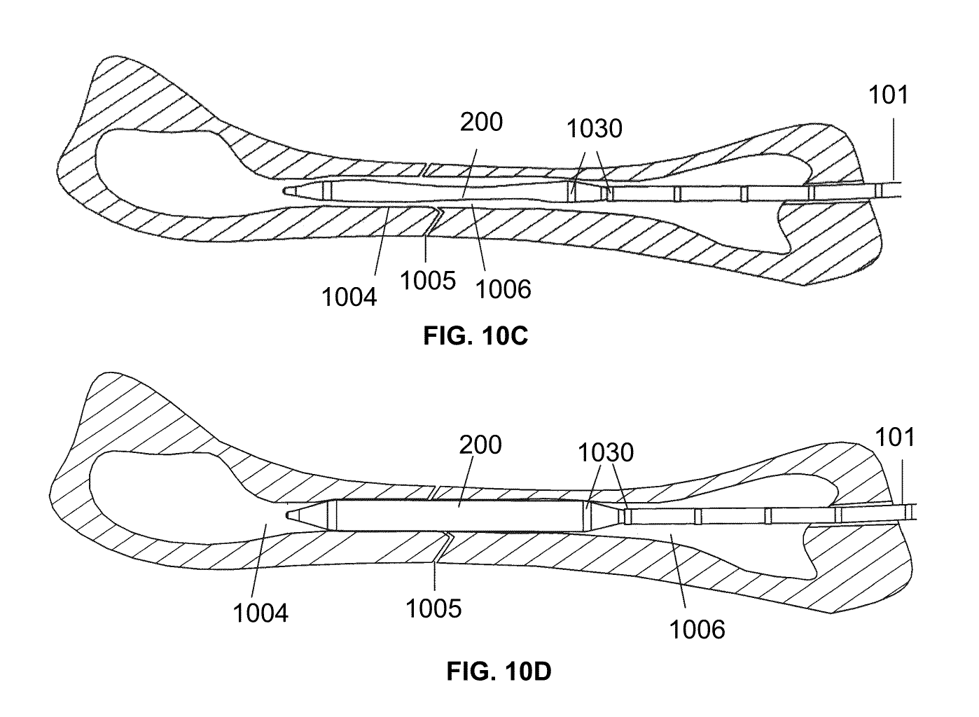

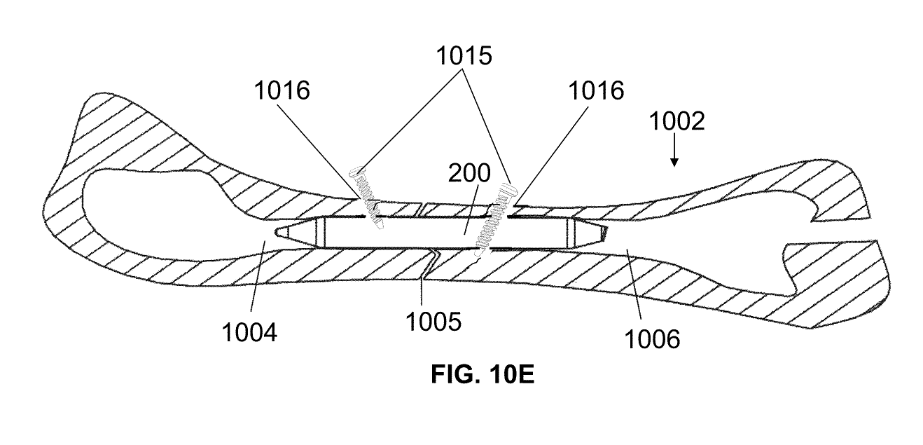

FIGS. 10A-10E, in combination with FIGS. 1 and 2, illustrate an embodiment of method steps for implanting an expandable portion of an intramedullary implant of the present disclosure within the intramedullary space of a weakened or fractured bone. A minimally invasive incision (not shown) is made through the skin of the patient's body to expose a fractured bone 1002. The incision may be made at the proximal end or the distal end of the fractured bone 1002 to expose the bone surface. Once the bone 1002 is exposed, it may be necessary to retract some muscles and tissues that may be in view of the bone 1002. As shown in FIG. 10A, an access hole 1010 is formed in the bone by drilling or other methods known in the art. In an embodiment, the access hole 1010 has a diameter of about 3 mm to about 10 mm. In an embodiment, the access hole 1010 has a diameter of about 3 mm.

The access hole 1010 extends through a hard compact outer layer 1020 of the bone into the relatively porous inner or cancellous tissue 1025. For bones with marrow, the medullary material should be cleared from the medullary cavity prior to insertion of the inventive device. Marrow is found mainly in the flat bones such as hip bone, breast bone, skull, ribs, vertebrae and shoulder blades, and in the cancellous material at the proximal ends of the long bones like the femur and humerus. Once the medullary cavity is reached, the medullary material including air, blood, fluids, fat, marrow, tissue and bone debris should be removed to form a void. The void is defined as a hollowed out space, wherein a first position defines the most distal edge of the void with relation to the penetration point on the bone, and a second position defines the most proximal edge of the void with relation to the penetration site on the bone. The bone may be hollowed out sufficiently to have the medullary material of the medullary cavity up to the cortical bone removed. There are many methods for removing the medullary material that are known in the art and within the spirit and scope on the presently disclosed embodiments. Methods include those described in U.S. Pat. No. 4,294,251 entitled “Method of Suction Lavage,” U.S. Pat. No. 5,554,111 entitled “Bone Cleaning and Drying system,” U.S. Pat. No. 5,707,374 entitled “Apparatus for Preparing the Medullary Cavity,” U.S. Pat. No. 6,478,751 entitled “Bone Marrow Aspiration Needle,” and U.S. Pat. No. 6,358,252 entitled “Apparatus for Extracting Bone Marrow.”

A guidewire (not shown) may be introduced into the bone 1002 via the access hole 1010 and placed between bone fragments 1004 and 1006 of the bone 1002 to cross the location of a fracture 1005. The guidewire may be delivered into the lumen of the bone 1002 and crosses the location of the break 1005 so that the guidewire spans multiple sections of bone fragments. As shown in FIG. 10B, the expandable portion 200 of the insertion catheter 101 for repairing a fractured bone, which is constructed and arranged to accommodate the guidewire, is delivered over the guidewire to the site of the fracture 1005 and spans the bone fragments 1004 and 1006 of the bone 1002. Once the expandable portion 200 is in place, the guidewire may be removed. The location of the expandable portion 200 may be determined using at least one radiopaque marker 1030 which is detectable from the outside or the inside of the bone 1002. Once the expandable portion 200 is in the correct position within the fractured bone 1002, a delivery system which contains a light-sensitive liquid is attached to the port 135. The light-sensitive liquid is then infused through the inner void 210 in the delivery catheter 101 and enters the inner cavity 235 of the expandable portion 200. This addition of the light-sensitive liquid within the expandable portion 200 causes the expandable portion 200 to expand, as shown in FIG. 10C. As the expandable portion 200 is expanded, the fracture 1005 is reduced. Unlike traditional implants, such as rods, that span the fracture site, the expandable portion 200 of the present disclosure does more than provide longitudinal strength to both sides of the fractured bone. In an embodiment, the expandable portion 200 having the design can be a spacer for reducing the fracture and for holding the fractured and compressed bones apart at the point of the collapsed fracture.

Once orientation of the bone fragments 1004 and 1006 are confirmed to be in a desired position, the light-sensitive liquid may be hardened within the expandable portion 200, as shown in FIG. 10D, such as by illumination with a visible emitting light source. In an embodiment, during the curing step, a syringe housing a cooling media may be attached to the proximal end of the insertion catheter and continuously delivered to the expandable portion 200. The cooling media can be collected by connecting tubing to the distal end of the inner lumen and collecting the cooling media via the second distal access hole. After the light-sensitive liquid has been hardened, the light source may be removed from the device. Alternatively, the light source may remain in the expandable portion 200 to provide increased rigidity. The expandable portion 200 once hardened, may be released from the delivery catheter 101 by known methods in the art. As shown in FIG. 10E, the hardened expandable portion remains in the fractured bone, and the insertion catheter is removed. In an embodiment, each surface of the expandable portion may be in contact with the bone. In an embodiment, at least a portion of a surface of the expandable portion may be in contact with the bone.

As shown in FIG. 10E, after the expandable portion 200 is in place, an intramedullary implant may be created by inserting one or more fasteners 1015, through the expandable portion 200 at a desired angle and anywhere along the length of the expandable portion 200, to secure the expandable portion 200 to the fractured bone fragment 1002. To insert a fastener 1015, a location along the length of the expandable portion 200 is selected by the user taking into consideration the patient's unique needs for bone stabilization. The location can be anywhere along the length of the expandable portion 200 since there are no predrilled holes in the expandable portion 200. After a location is selected, a hole 1016 can be drilled through the bone 1002 and expandable portion 200 at a desired angle and to a desired depth.

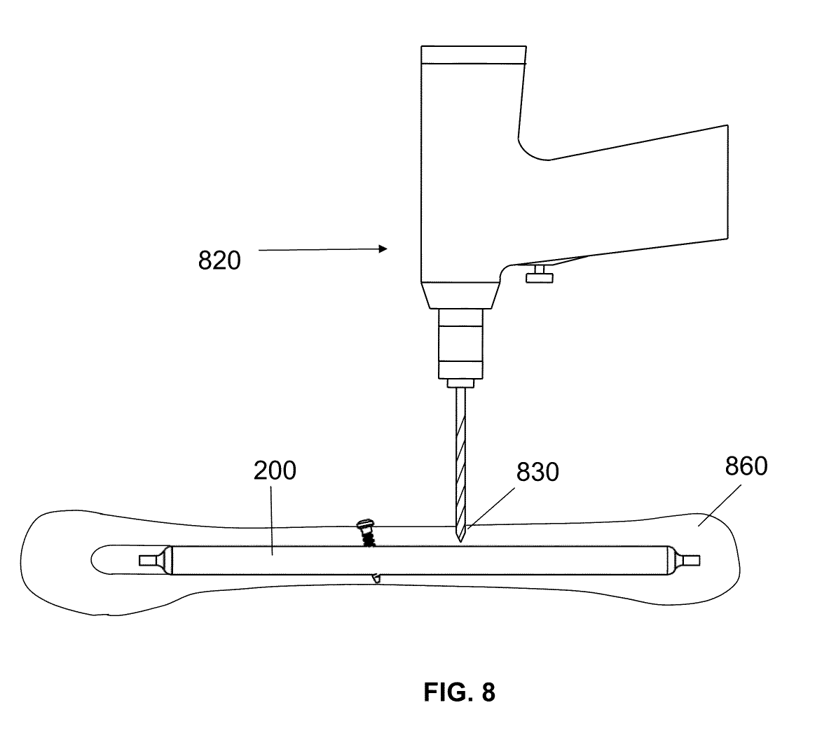

FIG. 8 is a side view of an embodiment of a drill 820 drilling a hole 830 through the weakened or fractured bone 860. In an embodiment, the drill 820 drills a hole (not shown) through expandable portion 200. In an embodiment, the bit portion of the drill 820 passes through a bone plate (not shown) to drill a hole (not shown) through the expandable portion 200. In an embodiment, the drill 820 may be a 2.5 mm drill. In an embodiment, the drill 820 may be any other commercially available drill. A fastener may then be inserted through the bone and the expandable portion at the desired angle and to the desired depth using a driver.

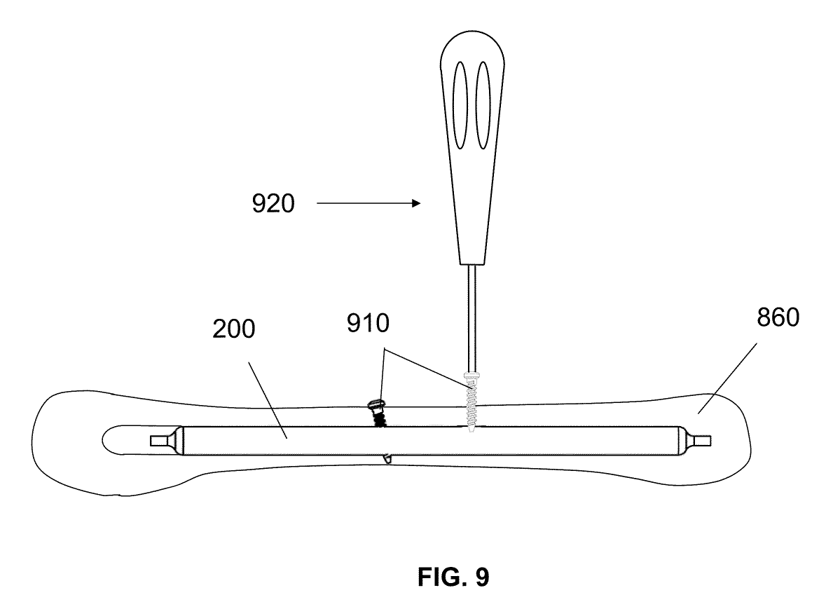

FIG. 9 is a side view of an embodiment of a fastener 910 being inserted through the weakened or fractured bone 860 and expandable portion 200 using a driver 920, resulting in an intramedullary implant of the present disclosure. In an embodiment, the driver 920 is a standard screw driver. In an embodiment, the driver 920 is a hammer. The fastener 910 may be secured to the bone 860 by directing the fastener 910 through the expandable portion 200. In an embodiment, the fastener 910 may be secured to the bone 860 by directing the fastener 910 through a bone plate (not shown) and then through the expandable portion 200. This procedure for inserting a fastener may be repeated as often as desired. It should be noted that the biocompatible nature of the expandable portion may reduce the likelihood of causing an adverse reaction if any fragments of the expandable portion become loose following the drilling of the expandable portion and the insertion of the fasteners into the expandable portion.

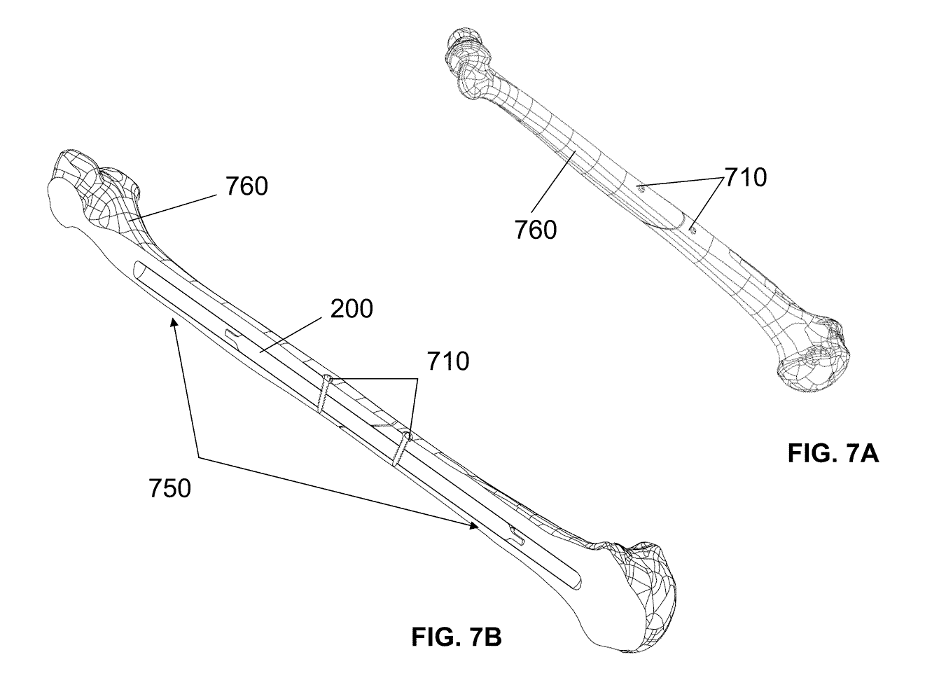

FIG. 7A and FIG. 7B are embodiments of an intramedullary implant 750 of the present disclosure implanted within the intramedullary space of a weakened or fractured bone 760. FIG. 7A is an isometric view of the intramedullary implant 750 positioned within the fractured or weakened bone 760. The intramedullary implant 750 includes expandable portion 200 and two fasteners 710. FIG. 7B is a sectional view of the intramedullary implant 750 supporting a weakened or fractured bone 760. In an embodiment, the expandable portion 200 includes two fasteners 710 securing the intramedullary implant 750 to the bone 760. Of course, the number of fasteners 710 securing the intramedullary implant 750 to the bone 760 may vary.

In an embodiment, a bone plate is used in conjunction with an intramedullary implant of the present disclosure. The bone plate may have any number of openings and can have a variety of shapes, sizes, and thicknesses for use in a variety of applications. The bone plate may have smooth openings, as well as, threaded openings. The smooth openings are generally used to receive a non-locking fastener and the threaded openings are generally used to receive a locking fastener. In an embodiment, the openings comprise pre-drilled holes. Non locking fasteners are generally used to draw the bone transversely toward the plate or to move the bone laterally through the use of compression plates. The bone plate may be positioned under soft tissue and on the exterior of the long bone and helps bridge the fractured portion of the long bone. In an embodiment, the bone plate is sufficiently strong to support a normal load on the long bone as the bone heals. In an embodiment, the bone plate has a stiffness substantially similar to a stiffness of the long bone. In an embodiment, the bone plate is made from a material that is non-toxic, non-antigenic and non-immunogenic. In an embodiment, the bone plate can be provided with a stiffness so that as the long bone heals, the bone plate allows the long bone to carry a larger load. In an embodiment, providing a bone plate that allows the long bone to carry a larger load as the bone heals avoids a reduction of bone mass of the bone. In an embodiment, the bone plate acts as a backing plate into which fasteners may be driven. In an embodiment, the when the distal end of the fasteners penetrate the outer surface of the expandable portion and are received by the bone plate, the bone plate helps hold the intramedullary implant in place.

The bone plate can be made from any material sufficiently strong to support the load placed on the bone while the bone heals. Examples of suitable materials include, but are not limited to titanium, stainless steel, ceramic polymeric materials such as hydroxyapatite, bioresorbable polymers, such as polylactic acid (PLA) or polycaprolactone (PCL), or other similar materials that allow the bone to be held together so that the bone can regenerate the tissue and regain most of the bone's original strength.

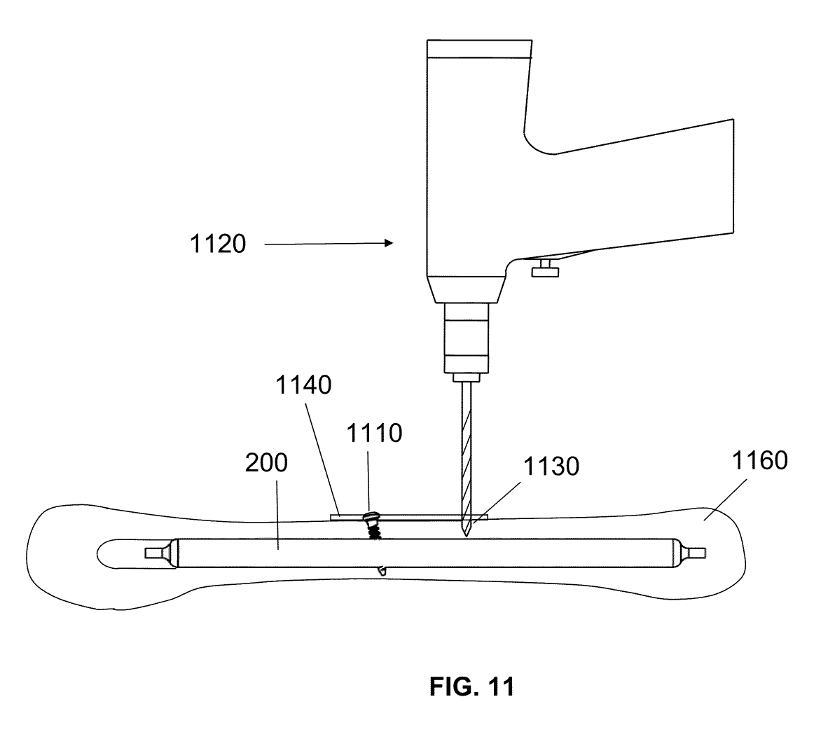

FIG. 11 is a side view of an embodiment of a drill 1120 drilling a hole 1130 through the weakened or fractured bone 1160. FIG. 11 shows a fastener 1110 driven through an external bone plate 1140, and penetrating the fractured bone 1160 and the expandable portion 200. In an embodiment, the drill 1120 drills a hole (not shown) through an expandable portion 200. In an embodiment, the drill 1120 may be a 2.5 mm drill. In an embodiment, the drill 1120 may be any other commercially available drill. A fastener may then be inserted through the bone and the expandable portion at the desired angle and to the desired depth using a driver. In an embodiment, only areas of the bone plate 1140 near or under the location of the fasteners 1110 are in contact with the fractured bone 1160, which results in less periosteal contact than is typical with conventional fixation devices. In an embodiment, less periosteal contact leads to better healing of the fractured bone 1160 due to the flexibility and micro-motion of the bone plate 1140.

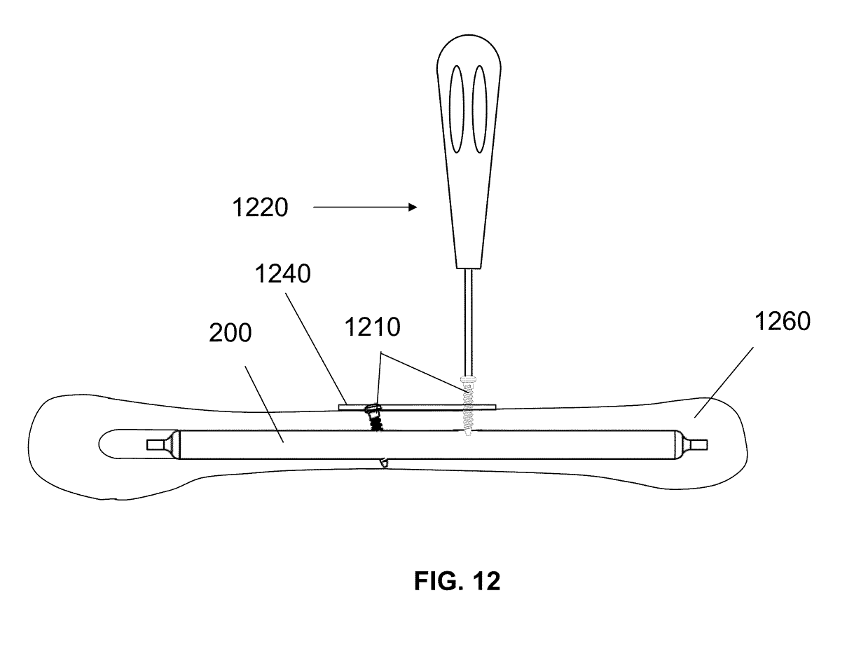

FIG. 12 is a side view of an embodiment of a fastener 1210 being inserted through the weakened or fractured bone 1260, an external bone plate 1240 and the expandable portion 200 using a driver 1220, resulting in an intramedullary implant of the present disclosure. In an embodiment, the driver 1220 is a standard screw driver. In an embodiment, the driver 1220 is a hammer. The fastener 1210 may be secured to the bone 1260 by directing the fastener 1210 through the bone plate 1240, through the fractured bone 1260, and through the expandable portion 200. The fastener 1210 can be angled into the expandable portion 1200 so as to cause compression between the proximal and distal sections of the expandable portion 200. In an embodiment, the fastener 1210 acts to pull the bone fragment pieces together, which can lead to improved alignment. This procedure for inserting a fastener may be repeated as often as desired. It should be noted that the biocompatible nature of the expandable portion may reduce the likelihood of causing an adverse reaction if any fragments of the expandable portion become loose following the drilling of the expandable portion and the insertion of the fasteners into the expandable portion. In an embodiment, only areas of the bone plate 1240 near or under the location of the fasteners 1210 are in contact with the fractured bone 1260, which results in less periosteal contact than is typical with conventional fixation devices. In an embodiment, less periosteal contact leads to better healing of the fractured bone 1260 due to the flexibility and micro-motion of the bone plate 1240.

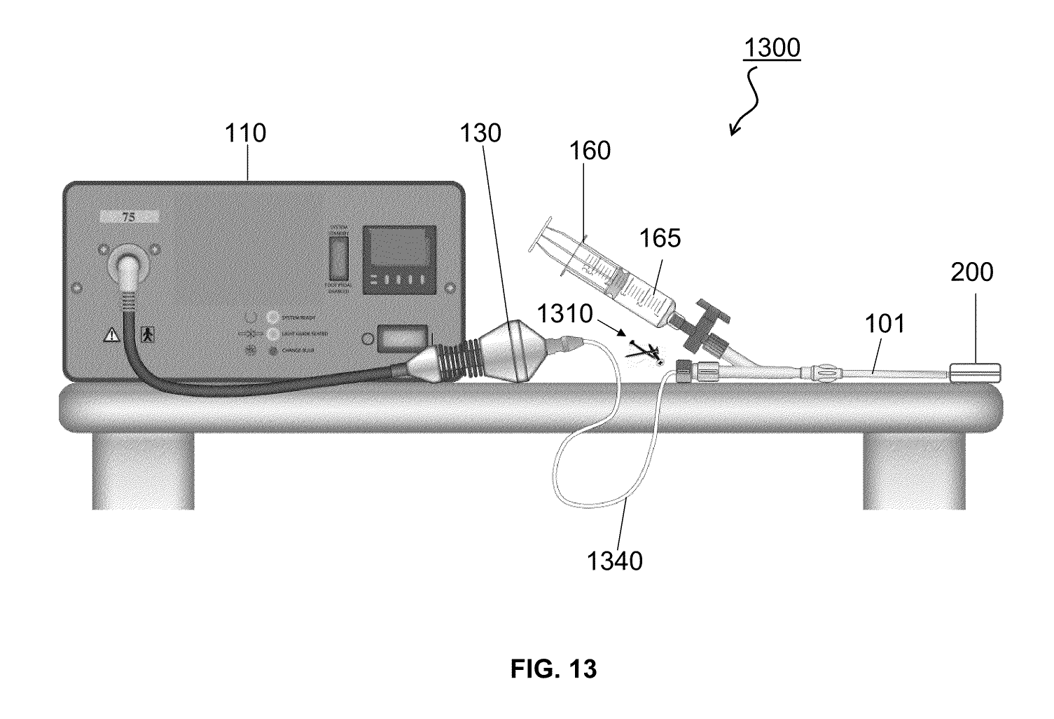

FIG. 13 is a schematic illustration of an embodiment of an intramedullary implant kit 1300 of the present disclosure. The kit 1300 includes a unit dose of a light sensitive liquid 165; a non-compliant expandable portion 200 releasably mounted on an insertion catheter 101, wherein the insertion catheter 101 has an inner void for passing the light-sensitive liquid 165 to the expandable portion 200, and an inner lumen; and at least one fastener 1310. In an embodiment, the light-sensitive liquid 165 is housed in syringe 160. In an embodiment, the syringe 160 maintains a low pressure during the infusion and aspiration of the light-sensitive liquid 165. In an embodiment, the syringe 160 maintains a low pressure of about 10 atmospheres or less during the infusion and aspiration of the light-sensitive liquid 165. In an embodiment, the kit 1300 further comprises an optical fiber 1340, wherein the optical fiber 1340 is sized to pass through the inner lumen of the insertion catheter 101 to guide a light into the expandable portion 200 to illuminate and cure the light-sensitive liquid 165. In an embodiment, an attachment system 130 communicates light energy from a light source 110 to the optical fiber 1340. In an embodiment, the light source 110 emits frequency that corresponds to a band in the vicinity of 390 nm to 770 nm, the visible spectrum. In an embodiment, the light source 110 emits frequency that corresponds to a band in the vicinity of 410 nm to 500 nm. In an embodiment, the light source 110 emits frequency that corresponds to a band in the vicinity of 430 nm to 450 nm. In an embodiment, the light-sensitive liquid 165 is a liquid monomer hardenable by visible light energy emitted by the light source 110.

In an embodiment, an intramedullary implant includes a non-compliant expandable portion having an outer surface and an inner cavity, wherein a hardened light-sensitive liquid is contained within the inner cavity of the expandable portion; and at least one fastener penetrating the expandable portion at a first location along the outer surface of the expandable portion and into the inner cavity of the expandable portion, wherein the at least one fastener penetrates the expandable portion at a user selected location anywhere along a length of the expandable portion, and wherein the at least one fastener penetrates the expandable portion at any angle relative to the expandable portion.

In an embodiment, a method for stabilizing a fractured bone includes penetrating the fractured bone to gain access to a medullary cavity of the fractured bone; inserting an expandable portion into the medullary cavity of the fractured bone; introducing a light-sensitive liquid monomer into the expandable portion so as to expand the expandable portion, wherein the light-sensitive liquid monomer is introduced into the expandable portion through at least one lumen of an insertion catheter releasably connected to the expandable portion, hardening the light-sensitive liquid monomer within the expandable portion so as to polymerize the light-sensitive liquid monomer; separating the insertion catheter from the expandable portion; and stabilizing the fractured bone, wherein the at least one fastener extends through an outer surface of the fractured bone, through an inner surface of the fractured bone, and into the expandable portion at any location along a length of the expandable portion, at any angle and to any penetration depth relative to the expandable portion.

In an embodiment, a method for realigning bone fragments includes providing an apparatus, wherein the apparatus includes a releasable expandable portion mounted on an insertion catheter, the insertion catheter having an inner void for passing a light-sensitive liquid, and an inner lumen for accepting a light-conducting fiber; positioning the expandable portion within a medullary canal of the bone fragments, wherein the expandable portion extends across/spans the bone fragments (fragment line); infusing the light-sensitive liquid into the inner void of the insertion catheter so that the light-sensitive liquid is delivered to the expandable portion and expands the expandable portion to a desired volume so as to realign the bone fragments; halting the infusing of the light-sensitive liquid; inserting a light-conducting fiber into the inner lumen of the insertion catheter so that the light-conducting fiber resides in the expandable portion; activating the light-conducting fiber to begin a polymerization process to polymerize the light-sensitive liquid within the expandable portion; removing the light-conducting fiber from the insertion catheter; releasing the expandable portion from the insertion catheter; selecting a location along a length of the expandable portion for insertion of at least one screw, wherein the selected location can be at any point along the length of the expandable portion; drilling a hole at the selected location through the bone fragment and the expandable portion; and inserting the at least one screw through the hole within the expandable portion, wherein the expandable portion having the at least one screw stabilizes the bone fracture.

In an embodiment, a method for bone fracture stabilization includes providing an apparatus for placement of an expandable portion within an intramedullary space spanning at least two fractured bone segments of a bone, wherein the apparatus includes a releasable expandable portion mounted on an insertion catheter, the insertion catheter having an inner void for passing a light-sensitive liquid; and an inner lumen for accepting a light-conducting fiber; inserting the expandable portion into the fractured bone to span the fractured bone segments; infusing the light-sensitive liquid into the inner void of the insertion catheter so that the light-sensitive liquid is delivered to the expandable portion; halting the infusing of the light-sensitive liquid; inserting the light-conducting fiber into the inner lumen of the insertion catheter so that the light-conducting fiber resides in the expandable portion; activating the light-conducting fiber to begin a polymerization process to polymerize the light-sensitive liquid within the expandable portion; removing the light-delivery fiber from the insertion catheter; releasing the expandable portion from the insertion catheter; selecting a location along the length of the expandable portion for insertion of at least one fastener, wherein the selected location can be at any point along the length of the expandable portion; drilling a hole at the selected location through the bone and the expandable portion; and inserting the at least one fastener through the hole within the expandable portion, wherein the expandable portion and the at least one fastener stabilizes the bone fracture. In an embodiment, the method is performed during a closed intramedullary nailing surgery.

All patents, patent applications, and published references cited herein are hereby incorporated by reference in their entirety. It will be appreciated that several of the above-disclosed and other features and functions, or alternatives thereof, may be desirably combined into many other different systems or application. Various presently unforeseen or unanticipated alternatives, modifications, variations, or improvements therein may be subsequently made by those skilled in the art.

What is claimed is:

1. An intramedullary implant comprising:
a non-compliant expandable portion having an outer surface and an inner cavity,
wherein a hardened light-sensitive liquid is contained within the inner cavity of the expandable portion; and
at least one fastener penetrating the expandable portion at a first location along the outer surface of the expandable portion and into the inner cavity of the expandable portion,
wherein the at least one fastener penetrates the expandable portion at a user selected location anywhere along a length of the expandable portion, and
wherein the at least one fastener penetrates the expandable portion at any angle relative to the expandable portion.

2. The implant of claim 1 wherein multiple fasteners penetrate the expandable portion at multiple locations along the outer surface of the expandable portion and into the inner cavity of the expandable portion at user selected locations anywhere along the length of the expandable portion.

3. The implant of claim 1 wherein multiple fasteners penetrate the expandable portion at multiple locations along the outer surface of the expandable portion and into the inner cavity of the expandable portion at user selected locations anywhere along the length of the expandable portion, and wherein the multiple fasteners penetrate the expandable portion from multiple directions and from multiple angles relative to the expandable portion.

4. The implant of claim 1 wherein the at least one fastener penetrates the expandable portion such that a distal end of the at least one fastener extends beyond the outer surface at a position that is opposite the first location of the outer surface of the expandable portion.

5. The implant of claim 1 wherein the at least one fastener is a non locking fastener.

6. The implant of claim 1 wherein the at least one fastener is a locking fastener.

7. The implant of claim 1 further comprising a bone plate having an opening and an internal thread in the opening for accepting the at least one fastener.

8. The implant of claim 1 wherein the non-compliant expandable portion is constructed from polyethylene terephthalate.

9. The implant of claim 1 wherein the light-sensitive liquid is hardened by energy emitted from a light source.

10. The implant of claim 1 wherein the light-sensitive liquid is hardened by exposure to visible light.

11. An intramedullary implant comprising:
a non-compliant expandable portion having an outer surface and an inner cavity, wherein the non-compliant expandable portion is sized for placement into a medullary canal of a bone;
a hardened light-sensitive liquid disposed within the inner cavity of the expandable portion; and
at least one fastener penetrating the expandable portion at a first location along the outer surface of the expandable portion and into the inner cavity of the expandable portion,
wherein the expandable portion, when placed into a medullary canal of a bone, is configured to accept the at least one fastener at a location anywhere along a length of the expandable portion, at any angle relative to the expandable portion and to any penetration depth.

12. An intramedullary implant kit comprising:
a unit dose of a light-sensitive liquid;
a non-compliant expandable portion releasably mounted on an insertion catheter, wherein the insertion catheter has an inner void for passing the light-sensitive liquid to the expandable portion, and an inner lumen; and
at least one fastener.

13. The kit of claim 12 wherein the non-compliant expandable portion is constructed from polyethylene terephthalate.

14. The kit of claim 12 further comprising an optical fiber, wherein the optical fiber is sized to pass through the inner lumen of the insertion catheter to guide a light into the expandable portion to illuminate and cure the light-sensitive liquid.

15. The kit of claim 12 wherein the light-sensitive liquid is a liquid monomer hardenable by visible light energy.

16. A method for stabilizing a fractured bone comprising:
penetrating the fractured bone to gain access to a medullary cavity of the fractured bone;
inserting an expandable portion into the medullary cavity of the fractured bone;
introducing a light-sensitive liquid monomer into the expandable portion so as to expand the expandable portion, wherein the light-sensitive liquid monomer is introduced into the expandable portion through at least one lumen of an insertion catheter releasably connected to the expandable portion;
hardening the light-sensitive liquid monomer within the expanded expandable portion so as to polymerize the light-sensitive liquid monomer;
separating the insertion catheter from the expandable portion;
positioning at least one fastener through the expandable portion; and
stabilizing the fractured bone,
wherein the at least one fastener extends through an outer surface of the fractured bone, through an inner surface of the fractured bone, and into the expandable portion at any location along a length of the expandable portion, at any angle and to any penetration depth relative to the expandable portion.

17. The method of claim 16 wherein the expandable portion is constructed from polyethylene terephthalate.

18. The method of claim 16 further comprising determining a location of the expandable portion within the medullary cavity of the fractured bone using at least one radiopaque marker positioned on the expandable portion.

19. The method of claim 18 wherein the at least one radiopaque marker is detectable from the outside of the fractured bone.

20. The method of claim 18 wherein the at least one radiopaque marker is detectable from the inside of the fractured bone.
